(19) United States
(12) Patent Application Publication
(10) Pub. No.: US 2024/0149044 A1
(43) Pub. Date: May 9, 2024

(54) PINCH CLAMP DEVICE AND ASSEMBLY

(71) Applicant: Becton, Dickinson and Company, Franklin Lakes, NJ (US)

(72) Inventor: Jason R. Stats, Layton, UT (US)

(21) Appl. No.: 17/982,313

(22) Filed: Nov. 7, 2022

(51) Int. Cl. A61M 39/28

(52) U.S. Cl. CPC A61M 39/28

(57) ABSTRACT

A pinch clamp device may include a first arm, which may include a first end, a second end, a first clamping surface, a first extension, and a set of first ledges proximate the first extension. The pinch clamp device may include a second arm, which may include a first end, a second end, a second clamping surface, a second extension, and a set of second ledges proximate the second extension and configured to align with the first clamping surface when the pinch clamp device is in a closed position. The set of first ledges may be configured to align with the second clamping surface when the pinch clamp device is in the closed position. The pinch clamp may include a hinge interconnecting the second end of the first arm and the second end of the second arm.

### BACKGROUND

The pinch clamp is a well-known type of one-piece plastic clamp which is used to close off intravenous tubing. The pinch clamp generally comprises a smooth, hard plastic material that is resilient and capable of controlled flexion to enable engagement and disengagement of the clamping surfaces.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a pinch clamp device that is designed for use in clamping or occluding intravenous tubing. In some embodiments, a pinch clamp device may include a first arm, which may include a first end, a second end, a first clamping surface, a first extension, and a set of first ledges proximate the first extension. In some embodiments, the pinch clamp device may include a second arm, which may include a first end, a second end, a second clamping surface, a second extension, and a set of second ledges proximate the second extension and configured to align with the first clamping surface when the pinch clamp device is in a closed position. In some embodiments, the set of first ledges may be configured to align with the second clamping surface when the pinch clamp device is in the closed position. In some embodiments, the pinch clamp device may include a hinge interconnecting the second end of the first arm and the second end of the second arm.

In some embodiments, the first extension may be U-shaped. In some embodiments, the second extension may be U-shaped. In some embodiments, the first end of the first arm may be a terminal end, which may include a first lip. In some embodiments, the first end of the second arm may be a terminal end, which may include a second lip. In some embodiments, the first lip may be configured to engage with the second lip when the pinch clamp device is in the closed position.

In some embodiments, the first clamping surface and the second clamping surface may be spaced apart when the pinch clamp device is in the closed position. In some embodiments, the set of first ledges may be offset from the set of second ledges when the pinch clamp device is in the closed position. In some embodiments, the first arm may include a set of first walls proximate the set of first ledges. In some embodiments, the second arm may include a set of second walls proximate the set of second ledges. In some embodiments, the first clamping surface may be configured to move outward to contact the set of second walls when the pinch clamp device is in the closed position configured to pinch an extension tube. In some embodiments, the second clamping surface may be configured to move outward to contact the set of first walls when the pinch clamp device is in the closed position configured to pinch the extension tube. In some embodiments, an outer surface of the first arm may include multiple rows of alphanumeric characters. In some embodiments, an outer surface of the second arm may include multiple rows alphanumeric characters.

In some embodiments, when the pinch clamp device is in the closed position, an outer surface of the first arm may slope downwardly to the first lip. In some embodiments, an outer surface of the pinch clamp device may include a protrusion, and pressure on the protrusion is configured to disengage the first lip with the second lip. In some embodiments, the first arm may include an elongated portion and a downwardly extending portion proximate the elongated portion. In some embodiments, the downwardly extending portion may be generally perpendicular to the elongated portion. In some embodiments, an outer surface of the downwardly extending portion may include the protrusion.

In some embodiments, a pinch clamp assembly may include the pinch clamp device and an extension tube extending through the pinch clamp device. In some embodiments, the extension tube may extend through the first extension and the second extension, and the first extension and the second extension are U-shaped. In some embodiments, when the pinch clamp device is in an open position, the extension tube may be trapped within the first extension and the second extension.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example pinch clamp device in an example open position, according to some embodiments;

FIG. 1B is a lower perspective view of the pinch clamp device in the open position, according to some embodiments;

FIG. 1C is another upper perspective view of the pinch clamp device, according to some embodiments;

FIG. 1D is an upper perspective view of the pinch clamp device in an example closed position, according to some embodiments;

FIG. 1E is a side view of the pinch clamp device in the open position, according to some embodiments;

FIG. 1F is a side view of the pinch clamp device in the closed position, according to some embodiments;

FIG. 1G is a transverse cross-sectional view of the pinch clamp, according to some embodiments;

FIG. 1H is a transverse cross-sectional view of the pinch clamp assembly, according to some embodiments;

FIG. 2 is an upper perspective view of the pinch clamp having an indented upper surface, according to some embodiments;

FIG. 3 is an upper perspective view of the pinch clamp having an example protrusion, according to some embodiments; and

FIG. 4 is an upper perspective view of the pinch clamp having an example stop surface, according to some embodiments.

### DESCRIPTION OF EMBODIMENTS

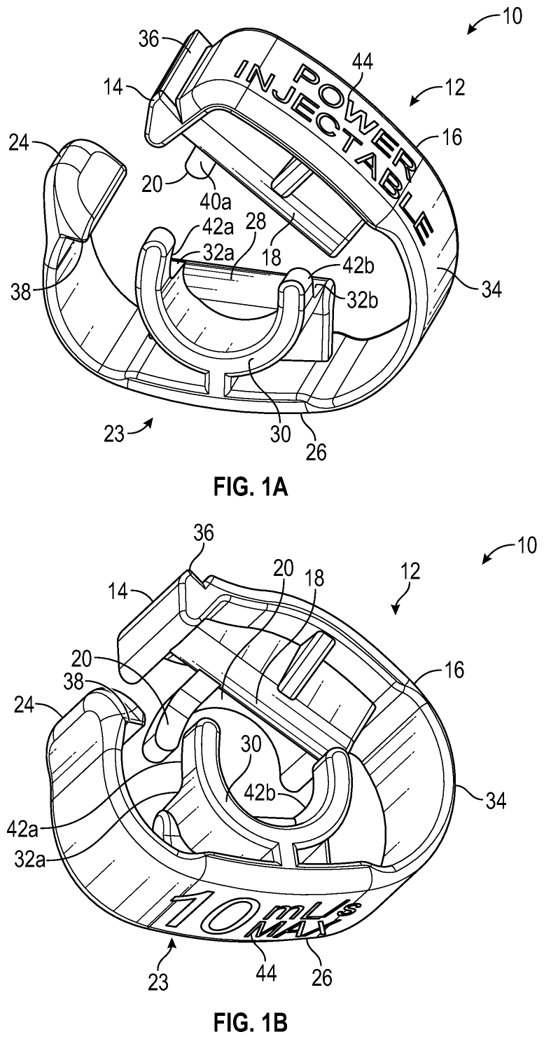

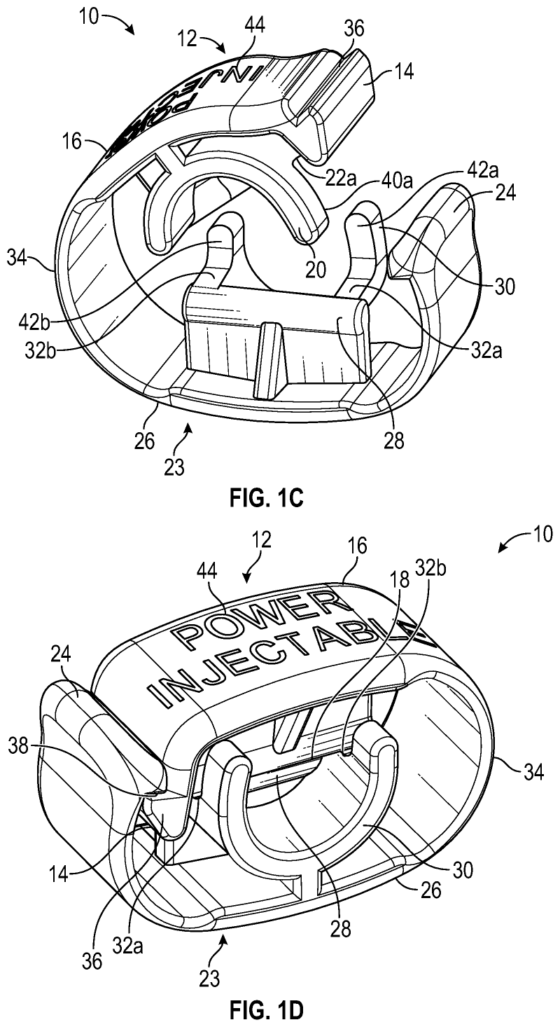

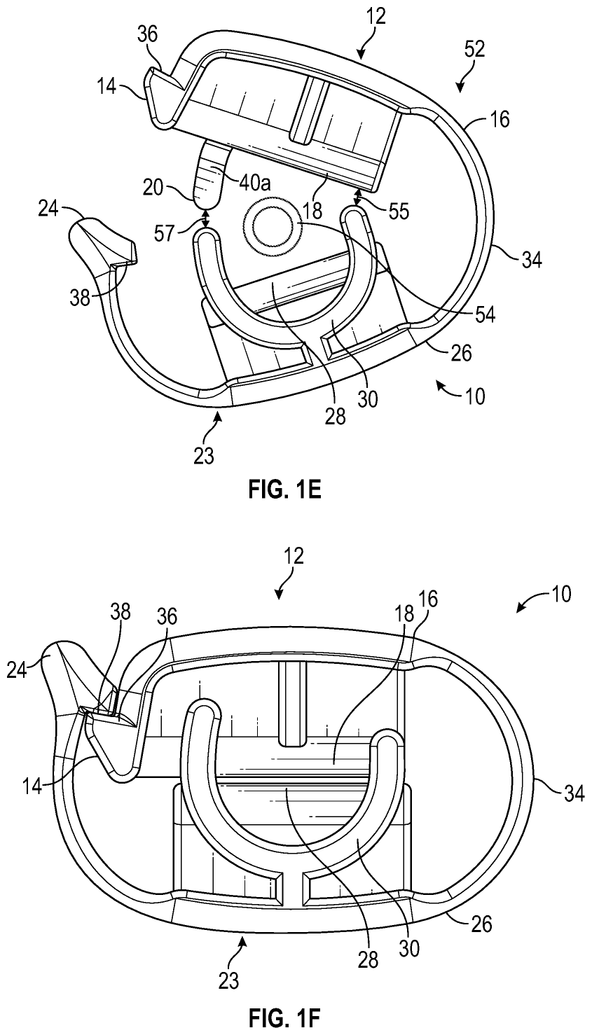

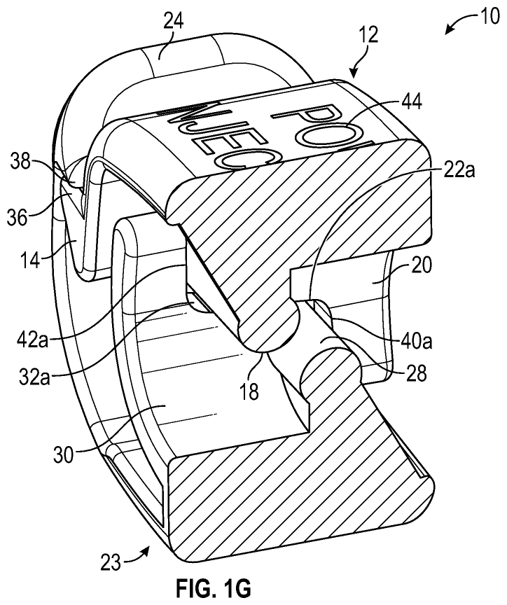

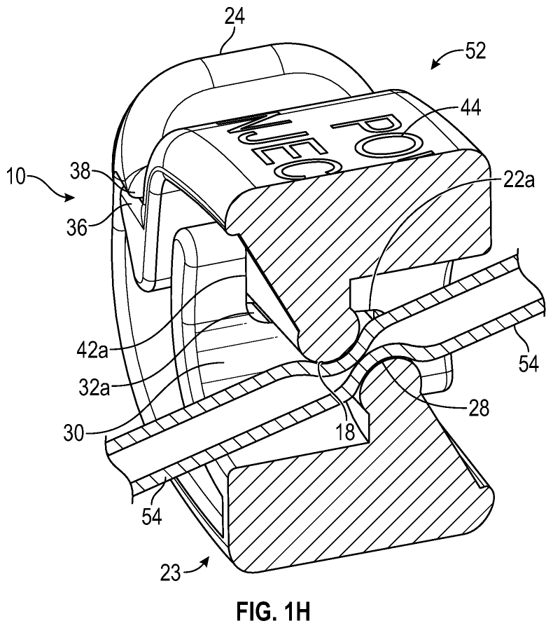

Referring now to FIG. 1A-1H, in some embodiments, a pinch clamp device 10 may include a first arm 12, which may include a first end 14, a second end 16, a first clamping surface 18, a first extension 20, and a set of first ledges 22a, 22b proximate the first extension 20. In some embodiments, the pinch clamp device 10 may include a second arm 23, which may include a first end 24, a second end 26, a second clamping surface 28, a second extension 30, and a set of second ledges 32a, 32b proximate the second extension 30 and configured to align with the first clamping surface 18 when the pinch clamp device 10 is in a closed position. In some embodiments, the set of first ledges 22a, 22b may be configured to align with the second clamping surface 28 when the pinch clamp device 10 is in the closed position. In some embodiments, the pinch clamp device 10 may include a hinge 34 interconnecting the second end of the first arm and the second end of the second arm.

In some embodiments, the first extension 20 and/or the second extension 30 may be U-shaped. In some embodiments, the first end 14 of the first arm 12 may be a terminal end, which may include a first lip 36. In some embodiments, the first end 24 of the second arm 23 may be a terminal end, which may include a second lip 38. In some embodiments, the first lip 36 may be configured to engage with the second lip 38 when the pinch clamp device 10 is in the closed position. In some embodiments, the first lip 36 and the second lip 38 may be configured to be engaged and disengaged with a single hand of a clinician.

In some embodiments, the first clamping surface 18 and the second clamping surface 28 may be spaced apart when the pinch clamp device 10 is in the closed position. In these embodiments, the set of first ledges 22a, 22b may be offset from the set of second ledges 32a, 32b when the pinch clamp device 10 is in the closed position. In further detail, the set of first ledges 22a, 22b may not be aligned with the set of second ledges 32a, 32b when the pinch clamp device 10 is in the closed position. In some embodiments, the first arm 12 may include a set of first walls 40a, 40b proximate the set of first ledges 22a, 22b. In some embodiments, the second arm 23 may include a set of second walls 42a, 42b proximate the set of second ledges 32a, 32b. In some embodiments, the first clamping surface 18 may be configured to move outward to contact the set of second walls 42a, 42b when the pinch clamp device 10 is in the closed position configured to pinch an extension tube. In some embodiments, the second clamping surface 28 may be configured to move outward to contact the set of first walls 40a, 40b when the pinch clamp device 10 is in the closed position configured to pinch the extension tube. Thus, in some embodiments, the set of first walls 40a, 40b and/or the set of second walls 42a, 42b may limit the first arm 12 and the second arm 23 spreading apart.

In some embodiments, an outer surface of the first arm 12 may include multiple rows of alphanumeric characters 44. In some embodiments, an outer surface of the second arm 23 may include multiple rows of alphanumeric characters 44. In some embodiments, a width of the first arm 12 and/or a width of the second arm 23 may be large enough to fit the multiple rows of alphanumeric characters 44 such that they are readable to the clinician. In some embodiments, a printable area on top and/or bottom of the pinch clamp device 10 may eliminate a need for a separate identification tag identifying the patient or a characteristic of the pinch clamp device 10.

In some embodiments, a pinch clamp assembly 52 may include the pinch clamp device 10 and an extension tube 54 extending through the pinch clamp device 10. In some embodiments, the extension tube 54 may extend through the first extension 20 and the second extension 30, and the first extension 20 and the second extension 30 are U-shaped. In some embodiments, when the pinch clamp device 10 is in an open position, the extension tube 54 may be trapped within the first extension 20 and the second extension 30, as illustrated, for example, in FIG. 1E. In some embodiments, when the pinch clamp device 10 is in the open position, a space 55 between the second extension 30 and the first clamping surface 18 and/or a space 57 between the first extension 20 and the second extension 30 may be less than an outer diameter of the extension tube 54 such that the extension tube 43 is trapped within the first extension 20 and the second extension 30 when the pinch clamp device 10 is in the open position. In some embodiments, the open position may correspond to a natural position of the pinch clamp device 10 when the first lip 36 is not engaged with the second lip 38.

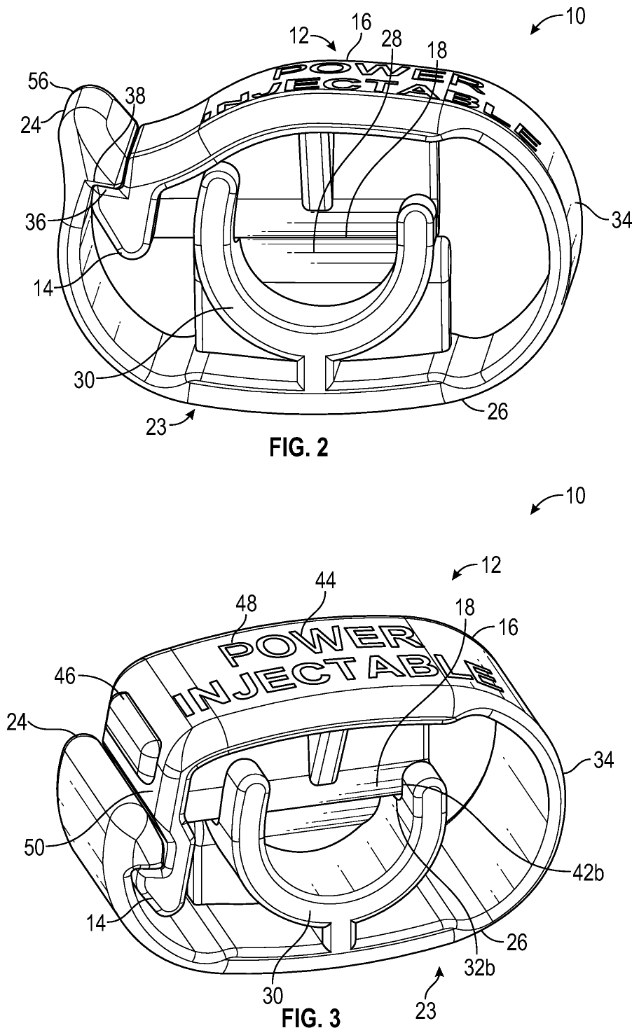

In some embodiments, the pinch clamp device 10 may provide patient comfort with a rounded design and/or few or no protruding features. In some embodiments, inner edges and/or outer edges of the pinch clamp device 10 may be smooth to facilitate patient comfort. Referring now to FIG. 2, the pinch clamp device 10 is illustrated having an upper surface that is indented, according to some embodiments. In some embodiments, the upper surface that is indented allows a larger push tab 56 without protruding out too far for patient comfort. In some embodiments, the push tab 56 may be disposed at the first end 24 of the second arm 23. In some embodiments, the pinch clamp device 10 may be sized and configured for use with a dialysis system, a peripheral intravenous catheter system or other suitable catheter system, or neonatal applications.

Referring now to FIG. 3, in some embodiments, when the pinch clamp device 10 is in the closed position, an outer surface of the first arm 12 may slope downwardly to the first lip 36. In some embodiments, an outer surface of the pinch clamp device 10 may include a protrusion 46, and pressure on the protrusion 46 is configured to disengage the first lip 36 with the second lip 38. In some embodiments, the first arm 12 may include an elongated portion 48 and a downwardly extending portion 50 proximate the elongated portion 48. In some embodiments, the downwardly extending portion 50 may be generally perpendicular to the elongated portion 48. In some embodiments, an outer surface of the downwardly extending portion 50 may include the protrusion 46.

In some embodiments, the protrusion 46 may function as a push button, and pushing on the protrusion 46 may move the pinch clamp device 10 to the open position. In some embodiments, pushing on an area of the pinch clamp device 10 other than the protrusion 46 may facilitate further engagement of the first lip 36 and the second lip 38, securing the pinch clamp device 10 further in the closed position. In some embodiments, the protrusion 46 may not extend past and may be interior to the push tab 56 may reduce a risk of the protrusion 46 irritating the patient. In some embodiments, the pinch clamp device 10 may include an oval shape. In some embodiments, the oval shape and the downwardly extending portion 50 may make the pinch clamp device 10 less likely to be accidentally compressed at an angle resulting in unclamping.

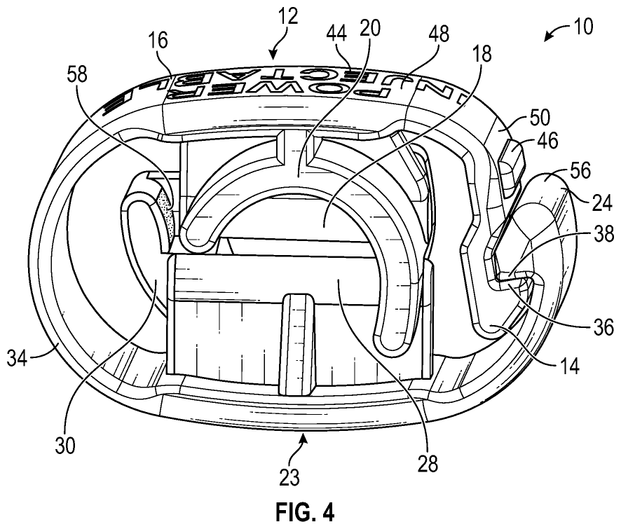

Referring now to FIG. 4, in some embodiments, the second arm 23 may include a stop surface 58. In some embodiments, the stop surface 58 may be disposed at an end of the second extension 30. In some embodiments, the stop surface 58 may be generally perpendicular and/or proximate to the second wall 42b and/or the second ledge 32b. In some embodiments, the stop surface 58 may be configured to prevent the first extension 20 from sliding when the protrusion 46 is pushed to unclamp the pinch clamp device 10.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A pinch clamp device, comprising:
a first arm, comprising a first end, a second end, a first clamping surface, a first extension, and a set of first ledges proximate the first extension;
a second arm, comprising a first end, a second end, a second clamping surface, a second extension, and a set of second ledges proximate the second extension and configured to align with the first clamping surface when the pinch clamp device is in a closed position, wherein the set of first ledges is configured to align with the second clamping surface when the pinch clamp device is in the closed position; and
a hinge interconnecting the second end of the first arm and the second end of the second arm.

2. The pinch clamp device of claim 1, wherein the first extension is U-shaped.

3. The pinch clamp device of claim 1, wherein the second extension is U-shaped.

4. The pinch clamp device of claim 1, wherein the first end of the first arm is a terminal end comprising a first lip, wherein the first end of the second arm is a terminal end comprising a second lip, wherein the first lip is configured to engage with the second lip when the pinch clamp device is in the closed position.

5. The pinch clamp device of claim 1, wherein the first clamping surface and the second clamping surface are spaced apart when the pinch clamp device is in the closed position.

6. The pinch clamp device of claim 1, wherein the set of first ledges is offset from the set of second ledges when the pinch clamp device is in the closed position.

7. The pinch clamp device of claim 1, wherein the first arm further comprises a set of first walls proximate the set of first ledges, wherein the second arm further comprises a set of second walls proximate the set of second ledges, wherein the first clamping surface is configured to move outward to contact the set of second walls when the pinch clamp device is in the closed position configured to pinch an extension tube, wherein the second clamping surface is configured to move outward to contact the set of first walls when the pinch clamp device is in the closed position configured to pinch the extension tube.

8. The pinch clamp device of claim 1, wherein an outer surface of the first arm comprises a plurality of rows of alphanumeric characters.

9. The pinch clamp device of claim 1, wherein an outer surface of the second arm comprises a plurality of rows alphanumeric characters.

10. The pinch clamp device of claim 1, wherein the first end of the first arm is a terminal end comprising a first lip, wherein when the pinch clamp device is in the closed position, an outer surface of the first arm slopes downwardly to the first lip.

11. The pinch clamp device of claim 1, wherein the first end of the first arm is a terminal end comprising a first lip, wherein the first end of the second arm is a terminal end comprising a second lip, wherein an outer surface of the pinch clamp device comprises a protrusion, wherein pressure on the protrusion is configured to disengage the first lip with the second lip.

12. The pinch clamp device of claim 11, wherein the first arm comprises an elongated portion and a downwardly extending portion proximate the elongated portion, wherein the downwardly extending portion is generally perpendicular to the elongated portion, wherein an outer surface of the downwardly extending portion comprises the protrusion.

13. A pinch clamp assembly, comprising:
a pinch clamp device, comprising:
a first arm, comprising a first end, a second end, a first clamping surface, a first extension, and a set of first ledges proximate the first extension;
a second arm, comprising a first end, a second end, a second clamping surface, a second extension, and a set of second ledges proximate the second extension and configured to align with the first clamping surface when the pinch clamp device is in a closed position, wherein the set of first ledges is configured to align with the second clamping surface when the pinch clamp device is in the closed position; and
a hinge interconnecting the second end of the first arm and the second end of the second arm; and
an extension tube extending through the pinch clamp device.

14. The pinch clamp assembly of claim 13, wherein the first extension is U-shaped.

15. The pinch clamp assembly of claim 13, wherein the second extension is U-shaped.

16. The pinch clamp assembly of claim 13, wherein the extension tube extends through the first extension and the second extension, wherein the first extension and the second extension are U-shaped.

17. The pinch clamp assembly of claim 16, wherein when the pinch clamp device is in an open position, the extension tube is trapped within the first extension and the second extension.

18. The pinch clamp assembly of claim 13, wherein the set of first ledges is offset from the set of second ledges when the pinch clamp device is in the closed position.

19. The pinch clamp assembly of claim 13, wherein the first arm further comprises a set of first walls proximate the set of first ledges, wherein the second arm further comprises a set of second walls proximate the set of second ledges, wherein the first clamping surface is configured to move outward to contact the set of second walls when the pinch clamp device is in the closed position configured to pinch the extension tube, wherein the second clamping surface is configured to move outward to contact the set of first walls when the pinch clamp device is in the closed position configured to pinch the extension tube.

20. The pinch clamp assembly of claim 13, wherein the first end of the first arm is a terminal end comprising a first lip, wherein the first end of the second arm is a terminal end comprising a second lip, wherein an outer surface of the pinch clamp device comprises a protrusion, wherein pressure on the protrusion is configured to disengage the first lip with the second lip, wherein the first arm comprises an elongated portion and a downwardly extending portion proximate the elongated portion, wherein the downwardly extending portion is generally perpendicular to the elongated portion, wherein an outer surface of the downwardly extending portion comprises the protrusion.
